(19) United States
(12) Patent Application Publication
(10) Pub. No.: US 2022/0218255 A1
(43) Pub. Date: Jul. 14, 2022

(54) KIT AND METHOD FOR MANAGING EMOTIONS IN KIDS

(71) Applicant: Danielle Jensen, Spring Hill, FL (US)

(72) Inventor: Danielle Jensen, Spring Hill, FL (US)

(21) Appl. No.: 17/572,601

(22) Filed: Jan. 10, 2022

Related U.S. Application Data
(60) Provisional application No. 63/135,111, filed on Jan. 8, 2021.

(51) Int. Cl. A61B 5/16; A61M 21/02; A63F 1/10

(52) U.S. Cl. CPC A61B 5/165; A63F 1/10; A61M 21/02

(57) ABSTRACT

A kit for regulating emotions and feelings by kids. The kit includes a main article that has avatars implemented to express different emotions, calming cards that include instructions to manage emotions by the kids, and calming articles that can be used by the kids to calm their emotions based on the instructions in the calming cards.

### CROSS-REFERENCE TO RELATED APPLICATIONS

This application claims priority from the U.S. provisional patent application Ser. No. 63/135,111, filed on Jan. 8, 2021, which is incorporated herein by reference in its entirety.

### FIELD OF INVENTION

The present invention relates to a kit and method to help adults teach emotional regulation to children, and more particularly, the present invention relates to kits for different age groups that can aid the kids in regulating their emotions.

### BACKGROUND

Kids like adults also feel diverse kinds of emotions, such as being happy, sad, angry, upset, jealous, and the like. However, the kids have unusual ways of expressing their emotions. These days, kids are spending most of the time indoors in families resulting in the development of complex feelings, such as being anxious. Parents lack the proper knowledge to help their children in managing such complex emotions. Social distancing and lockdowns during pandemics results in a negative effect on both adults and kids. However, kids find it more difficult than adults to cope up with their feelings. Working parents, because of their busy lifestyle, find it more difficult to help their kids in handling different situations.

Thus, an urgent need is there for an apparatus and method for kids that helps adults teach emotional regulation to children.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to a kit and method for managing emotions and feelings.

It is another object of the present invention that the kit and method allow adults to teach emotional regulation to children.

It is still another object of the present invention that the kit and methods are scientifically based.

It is a further object of the present invention that the kit and method can be adapted for kids of all ages.

It is still a further object of the present invention to relax an excessive pressure on the parents to handle complex emotions of their kids.

It is yet another object of the present invention that the kit can be used by children with disabilities such as SLD, ODD, ASD, ADHD & SPD.

It is yet a further object of the present invention that the kit is portable and discreet, easy to use, and convenient.

In one aspect, the kit can include a main article that allows kids to recognize their instant emotion and a calming article that can be selected based on the identified emotion from different calming articles, wherein the calming article allows the kids to regulate their emotions.

In one aspect, disclosed is a method for adults to teach emotional regulation to kids allowing the kids to manage their emotions, the method includes the steps for providing a kit for kids based on age group. Each kit includes a main article that can be used to recognize an instant emotion and the method further comprises a step of using a calming article to help calming an instant emotion.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 shows a main article of the disclosed kit for a 3-6 years age group, according to an exemplary embodiment of the present invention.

FIG. 2 shows a pouch of the kit, according to an exemplary embodiment of the present invention.

FIGS. 3a-3n show calming cards for 3-6 years age group, according to an exemplary embodiment of the present invention.

FIG. 4 shows a main article of the disclosed kit for a 7+ years age group, according to an exemplary embodiment of the present invention.

FIGS. 4a-4m show calming cards for the 7+ years age group, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

Disclosed is a kit that allows kids of all ages to self-regulate their emotions. Since the emotions can vary with age, as well as kids of different ages responding differently to the same stimulus, the kits can be designed for different age groups. For example, the two different kits can be designed for two age groups i.e., 3-6 years and 7+ years. The disclosed kit can help children to self-regulate their emotions while building a strong bond with parent. The disclosed kit can help adults, including parent, caretakers, healthcare professionals, and the like to teach emotional regulation to children. The disclosed kit can be a positive and gentle approach to parenting.

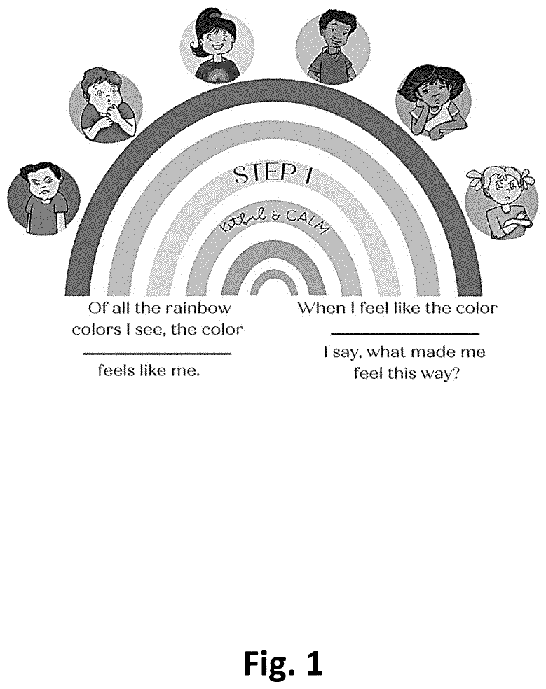

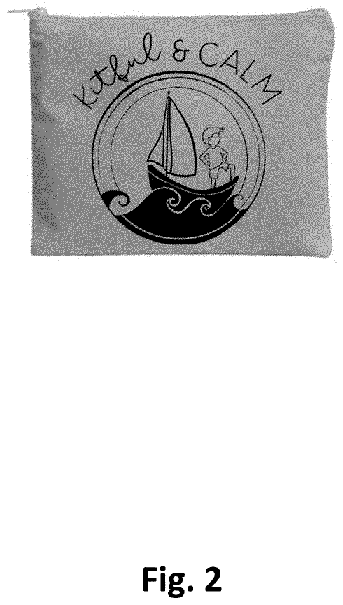

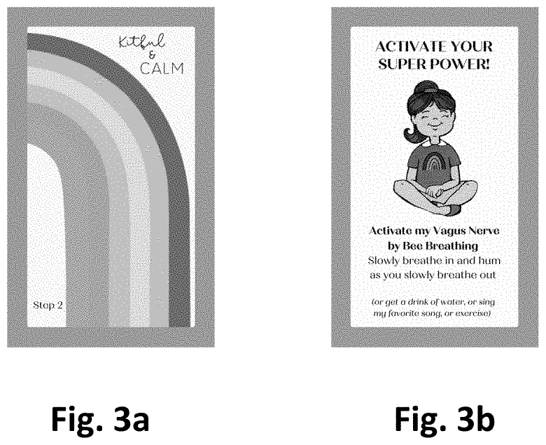

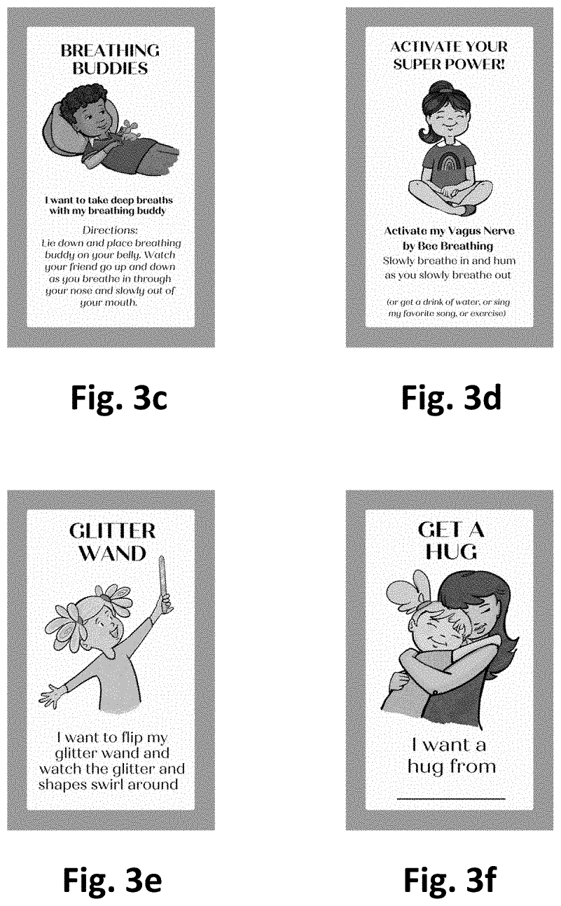

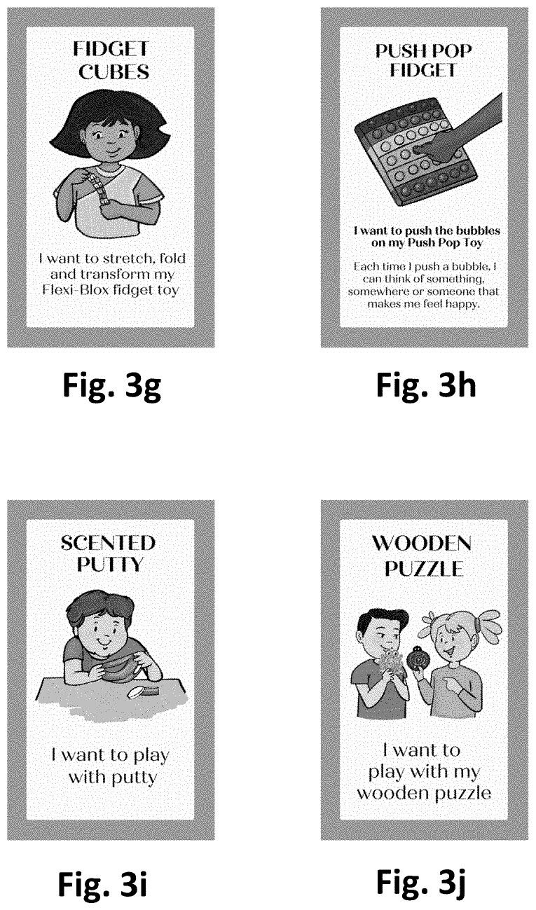

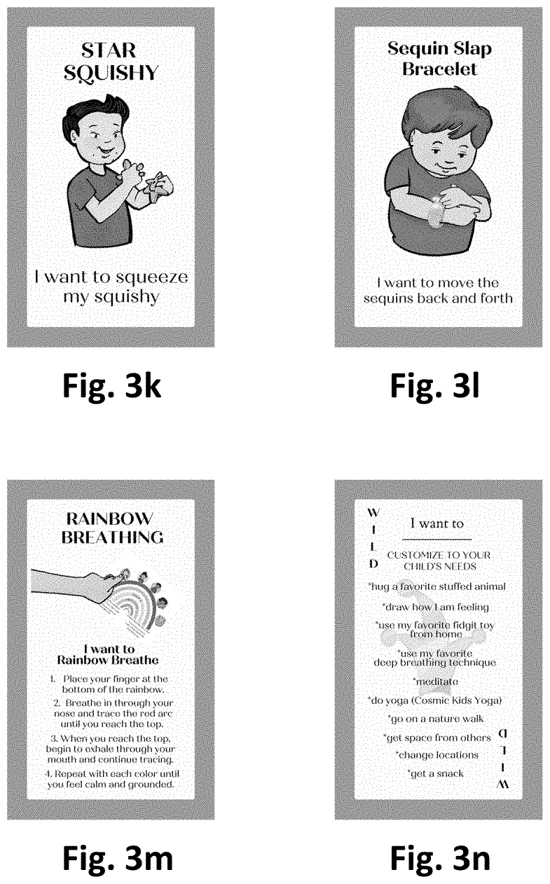

Referring to FIG. 1, which shows a main article of the kit designed for children in the age group of 3-6 years. The main article can be used to recognize an instant emotion by a kid. FIG. 2 shows a pouch of the kit that includes several calming articles that can be used by the kids to calm their emotions. The kit can include different calming articles including glitter wand, star stress ball, lightly scented putty, reversible sequin slap bracelet, push pop fidget, flexi-box fidget cubes, breathing buddy mini stuffed animal & wooden puzzle plus steps for breathing techniques, and more. The kit for 3-6 years of age can also include calming cards as shown in FIGS. 3a-3n. FIG. 3a shows a cover calming card and the calming cards can be secured in a key ring.

At first, a kid can recognize his instant feeling or emotion using the main article as shown in FIG. 1. The main article can be in a printed form that includes a poem and/or illustrations and rainbow colors which can help a kid to recognize his instant feelings or emotions. For each emotion or feeling, there can be a scientifically designed calming article that can help to regulate the associated emotion or feeling by the kids. Instruction to use the calming article can also be provided in a printed form using calming cards. For example, after recognizing by the kid an instant emotion or feeling that the kid is having using the main article, the kid can refer to the calming sheets that can include different ways to regulate the identified emotion using one or more of the calming articles. The main article, the instruction sheet, and the calming articles all can be provided in a pouch as shown in FIG. 2. The pouch can include a suitable fastening mechanism to secure the pouch to a user's wrist, stroller, and any other suitable support structure within reach of the kids. The instruction sheet can also be attached to a support member, such as a key ring. Preferably about 12 calming cards can be provided in a kit.

Again, referring to FIG. 1, which shows the main article as a printed sheet having illustrations of kids (emoticons or avatars) with different emotions, such as angry, surprised, happy, laughing, sad, jealous, and like kid's emotions. Also, a rainbow can be provided on the printed sheet. The user i.e., the kid or child can find at least one avatar from the printed sheet that matches with his instant feeling or emotion. For example, if the user is sad, he can select the sad avatar illustrated on the printed sheet. The illustration of kids with emotions in the main article is also herein referred to as emoticons or avatars.

The calming cards can be used by kids that include instructions to calm the emotions with or without using the calming articles. The calming card can show instruction on how to regulate the emotion and if required how to use a calming article. The calming articles can be used to calm, pacify, or regulate in any other way the instant emotions or feelings. For example, watching the glitter fall can distract from being upset, hugging the stuffed animal when sad, or placing the stuffed animal on the belly to help belly breathe, and watching the animal go up and down are some of the ways of how the calming article can be used by the kids to self-regulate their emotions. The main article can also have instruction in the form of a poem to help the kid recognize an illustrated avatar or emoticon on the printed sheet that best matches his instant feeling or emotion.

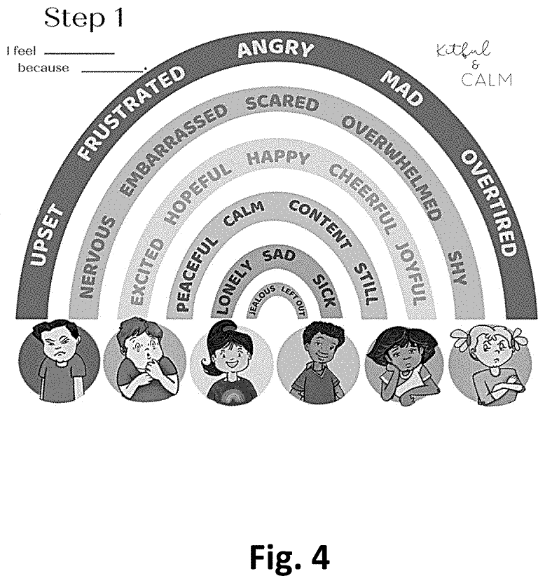

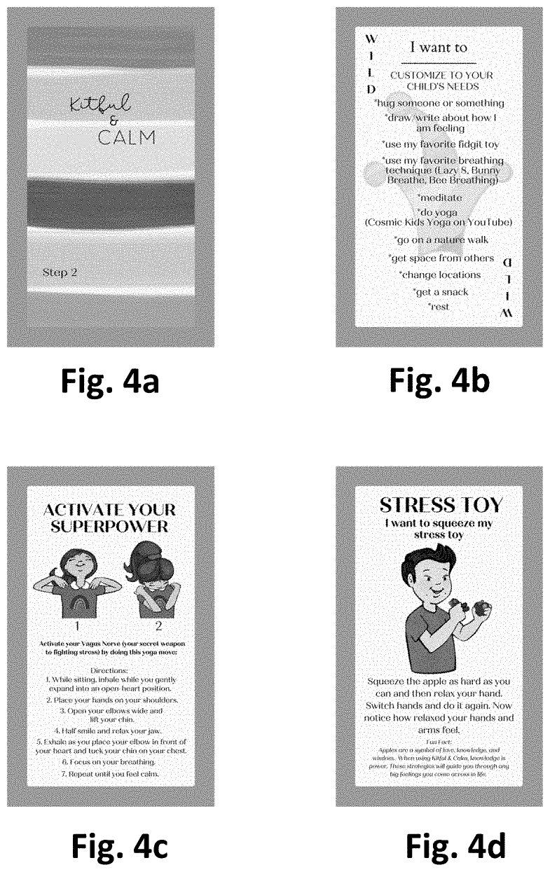

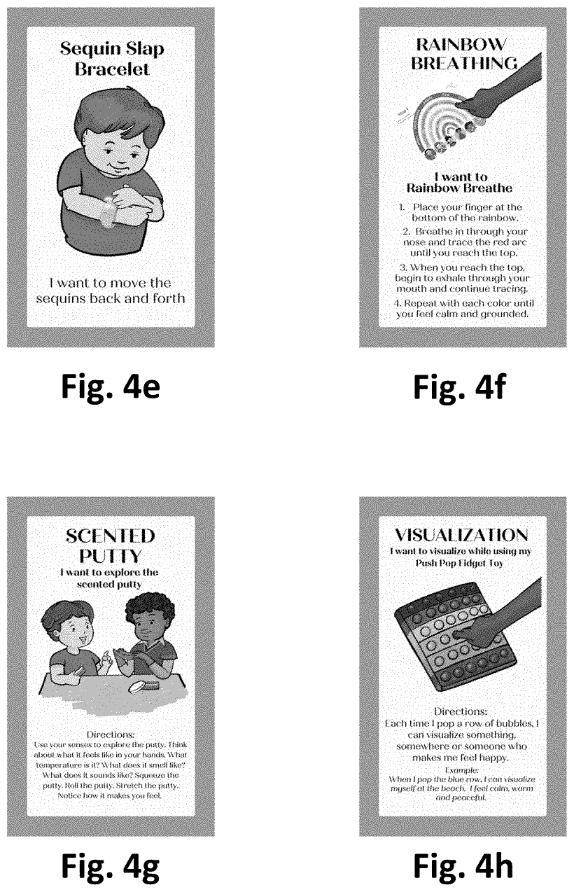

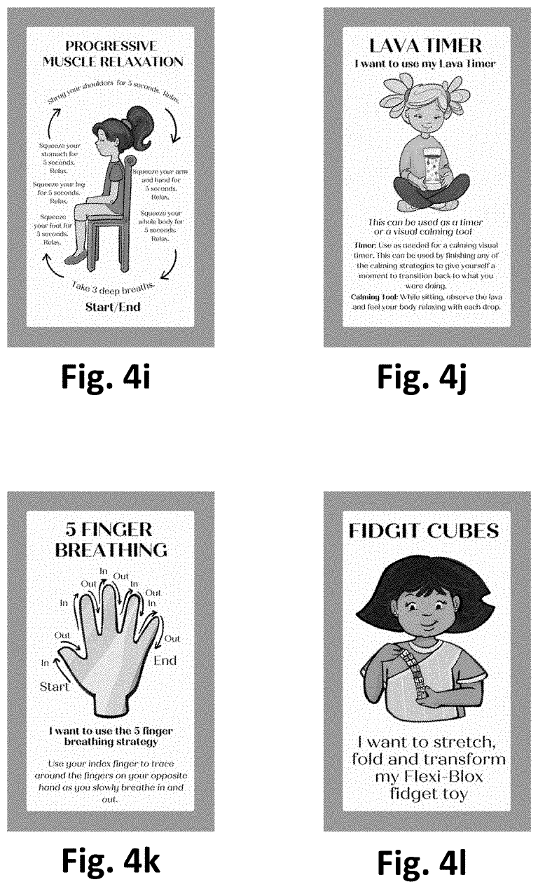

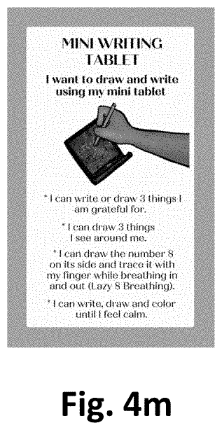

Referring to FIG. 4 which shows a main article for the kit designed for the age group of 7+ years. FIGS. 4a-4m show the calming cards for the kit i.e., the kit for 7+ years. The calming tools for the 7+ years kit can include a lava timer, apple stress ball, lightly scented putty, reversible sequin slap bracelet, push pop fidget, full-size flexi-box fidget cubes & mini writing tablet plus steps for breathing techniques, progressive muscle relaxation and more. This kit can also be provided in a sapphire blue 8×11 canvas wristlet bag. The main article can be a laminated 5×7 card for children to identify their feelings. Twelve calming cards with research-based techniques on a key ring to easily flip through. All of the calming tools to go along with the calming cards.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A method for regulating emotions and feeling by kids, the method comprising the steps of:
providing a kit comprising:
a main article comprising a plurality of avatars implemented to express a plurality of emotions;
a plurality of calming cards, each calming card of the plurality of calming cards comprises an instruction to manage an emotion; and
a plurality of calming articles, wherein each calming article of the plurality of calming articles can be used to calm an emotion.

2. The method according to claim 1, wherein the kit further comprises a pouch enclosing the main article, the plurality of calming cards, and the plurality of calming articles.

3. The method according to claim 2, wherein the pouch has a fastening member configured to allow securing the pouch to a supporting structure.

4. The method according to claim 3, wherein the kit further comprises a keyring securing the plurality of calming articles.
